(19) United States
(12) Patent Application Publication
(10) Pub. No.: US 2008/0177197 A1
(43) Pub. Date: Jul. 24, 2008

(54) Method and apparatus for quantitatively evaluating mental states based on brain wave signal processing system

(76) Inventors: KooHyoung Lee, Sunnyvale, CA (US); Stanley Yang, Los Altos, CA (US)

Correspondence Address: DLA PIPER US LLP, 2000 UNIVERSITY AVENUE, E. PALO ALTO, CA, 94303-2248, US

(21) Appl. No.: 11/656,828

(22) Filed: Jan. 22, 2007

(51) Int. Cl. A61B 5/04

(52) U.S. Cl. 600/545

(57) ABSTRACT

A noise-free portable EEG system is provided. The system has hardware and software and can evaluate mental state quantitatively. The quantitative data of mental states and their levels can be applied to various areas of brain-machine interface including consumer products, video game, toys, military and aerospace as well as biofeedback or neurofeedback.

### FIELD

The field relates generally to an apparatus and method for quantitatively evaluating mental states.

### BACKGROUND

There are many available ways to detect brain waves and utilize them as control signals as well as diagnostic tools. However, there are still many barriers to measuring brain waves without noise, especially, outside of a well-controlled laboratory environment. Typically, brain waves can be detected and utilized in the laboratories where environmental and electromagnetic noises are strictly controlled and only static condition, for the patient or subject whose brain waves are being measured, is that the patent or subject should not move. Such idea settings do not exist outside of the laboratory so that these systems cannot be used to reliable measure the brain waves of a user. In addition, typical sensor placement requires a special treatment to the head since most currently used electrodes for measuring the brain waves require either electrodes that are wet with gel or needle electrodes.

Such idea settings do not exist outside of the laboratory so that these systems cannot be used to reliable measure the brain waves of a user in a non-laboratory environment. In addition, the special treatment of a head to use the laboratory electrodes is not practical in a non-laboratory environment. Thus, it is desirable to provide an apparatus and method that overcomes these limitations of typical brain wave measurement systems and it is to this end that the present invention is directed.

### SUMMARY OF THE INVENTION

The apparatus may include a neuro headset that includes one or more dry active electrodes that measure the brain waves of a user wearing the headset without wet electrodes. The apparatus may be incorporated into a system that provides a human/machine interface using the neuro headset, additional hardware and software. For example, an illustrative system is a system for controlling a toy using the brain waves of the user as is described below in more detail. In the system, the hardware detects brain waves, filters out noises and amplifies the resultant signal. The software processes the brain wave signal, displays the mental state of the user based on the analysis of the brain wave signals and generates control signals that can be used to control a device, such as a toy.

### BRIEF DESCRIPTION OF THE DRAWINGS

FIG. 1A illustrates an example of an apparatus for quantitatively evaluating mental states that is being used to control the actions of a toy;

FIG. 1B illustrates an exemplary implementation of the dry-active electrode used in the apparatus of FIG. 1;

FIGS. 2A and 2B illustrate a neuro headset that is part of the apparatus shown in FIG. 1A;

FIGS. 3A and 3B illustrate further details of the apparatus shown in FIGS. 1A, 2A and 2B;

FIG. 4 illustrates an implementation of a system for controlling a toy using the apparatus for quantitatively evaluating mental states that includes the neuro headset shown in FIGS. 2A, 2B, 3A and 3B, other hardware and software;

FIGS. 5A and 5B illustrate more details of the hardware of the system shown in FIG.

FIG. 6 illustrates an exemplary circuit implementation of the digital portion of the hardware shown in FIG. 4;

FIG. 7 illustrates an exemplary circuit implementation of the power regulation portion of the hardware shown in FIG. 4;

FIG. 8A illustrates more details of an analog portion of the dry-active electrodes;

FIG. 8B illustrates more details of the analog portion of the dry-active electrodes;

FIG. 9 illustrates an exemplary circuit implementation of the analog EEG signal processing portion shown in FIG. 5;

FIG. 10A is a block diagram of the analog EOG signal processing portion shown in FIG. 5;

FIG. 10B illustrates an exemplary circuit implementation of the analog EOG signal processing portion shown in FIG. 5;

FIG. 11 illustrates an example of the operation of the software that is part of the shown in FIG. 4;

FIG. 12 illustrates further details of the data processing process of FIG. 11;

FIG. 13 illustrates a flowchart of the data processing steps; and

FIG. 14 illustrates an example of the graphical displays of the mental state of the user.

### DETAILED DESCRIPTION OF ONE OR MORE EMBODIMENTS

The apparatus and method are particularly applicable to a system for controlling a toy using the brain waves of the user and it is in this context that the apparatus and method will be described below for illustration purposes. However, it will be appreciated that the apparatus and method may be used for applications other than controlling a toy and in fact can be used in any application in which it is desirable to quantitatively evaluate the brain waves of a user and provide a human-machine interfaces and/or neuro-feedback based on the quantitatively evaluation of the brain waves. For example, apparatus and method may be used to control a computer or computer system, game console, etc. As another example, the apparatus and method may be implemented and integrated into a pilot's helmet with a brain wave monitoring system built into the helmet wherein the dry sensors can monitor pilot's brain waves during flight and, if the pilot loses consciousness during flight, the apparatus can detect the loss of consciousness and perform one or more actions such as engaging the auto-pilot system and providing emergency treatment/alert to the pilot (such as oxygen or vibration) which can save the plane and the life of the pilot. The apparatus and method may also be implemented as a headband-style patient brain wave monitoring system where the EEG of the patient is monitored with the dry sensors which is easy to use and user-friendly to patients and the brain wave can be transmitted using wireless method (such as Bluetooth) or wired method to a remote device that can record/display the EEG signals of the patient. As another example, the apparatus and method can be implemented and integrated into a combat helmet with a brain wave monitoring system wherein the dry sensors can monitor brain wave of soldiers and send warning signals to the soldier (a sound alert, a visual alert or a physical alert such as a shock) if the soldier loses consciousness or falls asleep during a task.

As another example, the apparatus and method can be incorporated into safety gear for an employee since many accidents happen in the factory when workers lose mental concentration on the task. The safety gear, which has the forms of headband, baseball cap or hard hat with the dry sensors and EEG system, can stop a machine if the worker's mental concentration level goes down to the designated level to prevent accidents and protect the employee.

As another example, the apparatus and method can be incorporated into a sleep detector for drivers wherein the detector is a headband-style, headset style or baseball cap style that has a brain wave monitoring system with dry sensors that can detect a driver's drowsiness or sleep (based on the brain wave) and provide warning signals to the driver or stimulus to wake the driver up.

As yet another example, the apparatus and method can be implemented in a stress management system that has a headband style, headset style or baseball cap style brain wave monitoring system with the dry sensors that can be connected to a computing device, such as a PC, PDA or mobile phone, in order to monitor mental stress level during a job and record those stress levels. The above examples of the applications for the apparatus and method are not exhaustive. To illustrate the apparatus and method, an exemplary system for controlling a toy using the apparatus and method is now described.

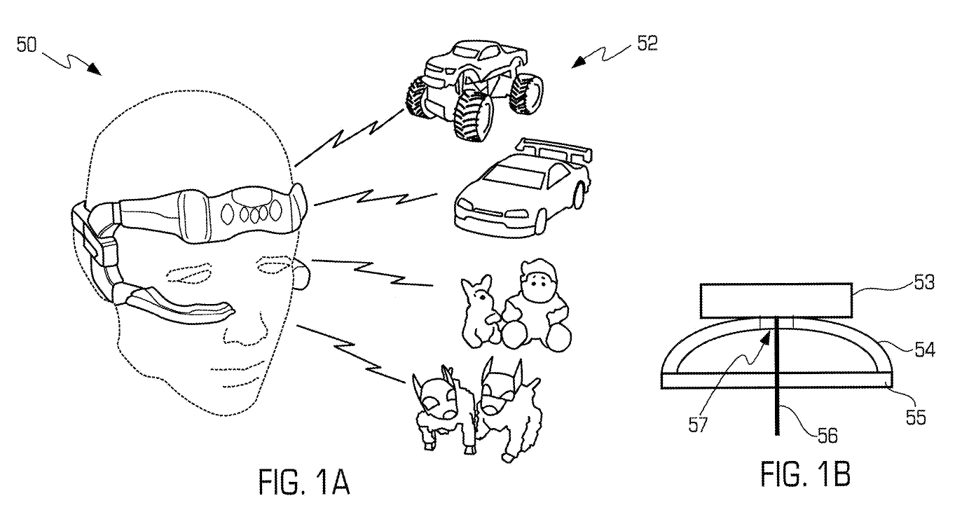

FIG. 1A illustrates an example of an apparatus for quantitatively evaluating mental states that is being used to control the actions of a toy. The apparatus may include a neuro headset 50 that may be placed onto the head of a user as shown in FIG. 1A. The neuro headset may include various hardware and software that permits the user, when wearing an powered up headset, to control a device wirelessly such as a toy 52 based on the brain waves of the user. The apparatus may in fact be used to control a plurality of different toys, such as a truck, car, a figure or a robotic pet provided that the apparatus has the proper information to generate the necessary control signals for the particular toy. The headset 50 may include one or more dry-active electrodes (sensors) that are used to detect the brain waves of the user. The one or more electrodes may be adjacent the forehead of the user and/or adjacent the skin behind the ears of the user.

FIG. 1B illustrates an exemplary implementation of a mechanical portion of the dry-active electrode used in the apparatus of FIG. 1. The sensor may also comprise an electronic portion shown in more detail in FIG. 8 wherein the electronic portion can be separated from the mechanical portion. The dry-active electrode/sensor has a silver/silver chloride (Ag/AgCl) electrode 53 and a spring mechanism 54, such as a thin metal plate, that is attached to a base 55 that may be a non-conductive material. The spring mechanism permits the electrode 53 to be biased towards a user by the spring mechanism when the sensor is placed against the skin of the user. The electrode may also have a conductive element 56, such as a wire, that receives the signals picked up by the electrode and transmits the signal to the analog processing part described below. The spring mechanism 54 may have a hole region 57 with non-conductive material that isolates the conductive element 56 from the spring mechanism 54. The dry-active electrodes and module used in the exemplary implementation of the apparatus are described in more detail in co-pending U.S. patent application Ser. No. 10/585,500 filed on Jul. 6, 2006 that claims priority from PCT/KR2004/001573 filed on Jun. 29, 2004 which in turn claims priority from Korean Patent Application Serial No. 10-2004-0001127 filed on Jan. 8, 2004 which are all commonly owned and incorporated herein by reference.

The apparatus may include one or more pieces of software (executed by a processing unit within the headset, embedded in a processing unit in the headset or executed by a processing unit external to the headset) that perform one or more functions. Those functions may include signal processing procedures and processes and processes for quantitatively determine the mental states of the user based at least in part on the brain waves of the user. The determined mental states can be expressed as attention, relaxation, anxiety, drowsiness and sleep and the level of each mental state can be determined by the software and expressed with number from 0 to 100, which can be changed depending on applications. In addition to the toy control application shown in FIG. 1, the apparatus may also be used for various human-machine interfaces and neuro-feedback.

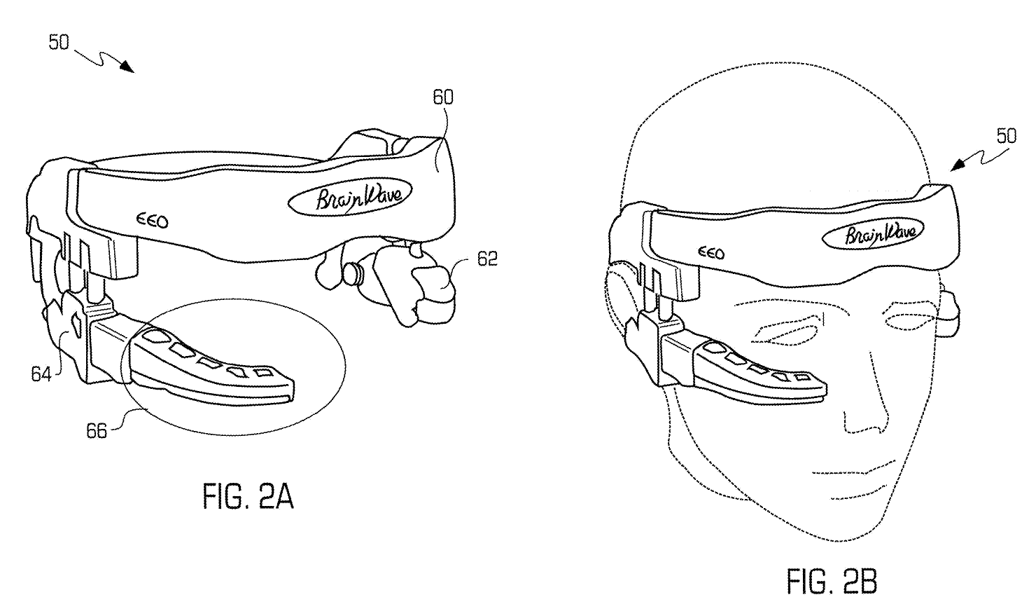

FIGS. 2A and 2B illustrate a neuro headset 50 that is part of the apparatus shown in FIG. 1 wherein FIG. 2A is a perspective view of the headset and FIG. 2B is a perspective view of the headset when worn by a user. The headset may have a front portion 60 a first side portion 62 and a second side portion 64 opposite of the first side portion. When worn by a user as shown in FIG. 2B, the front portion 60 rests against the forehead of the user so that one or more dry sensors in the front portion rest against the forehead of the user. The first and second side portions 62, 64 fit over the ears of the user. The headset may further include a boom portion 66 that extends out from the second side portion 64. The boom portion 66 may include a eye movement sensor that permits the headset to measure or detect the eye movement of the user when the headset if active.

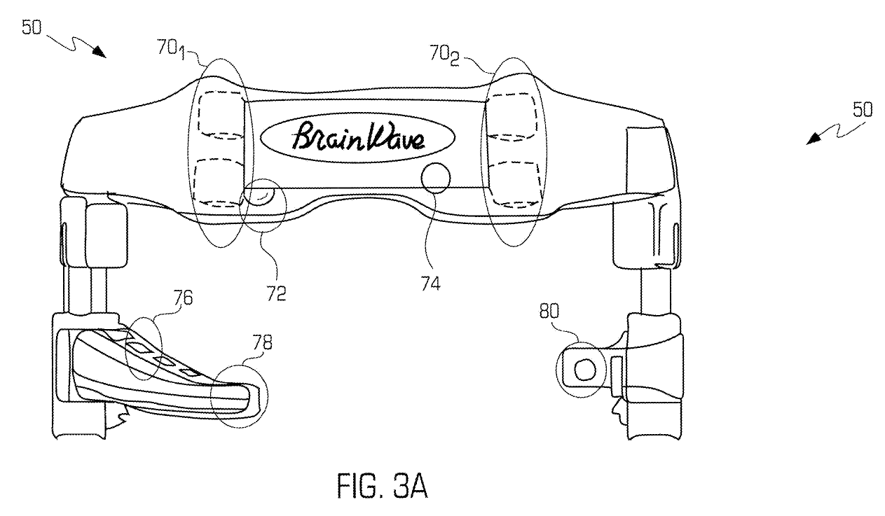

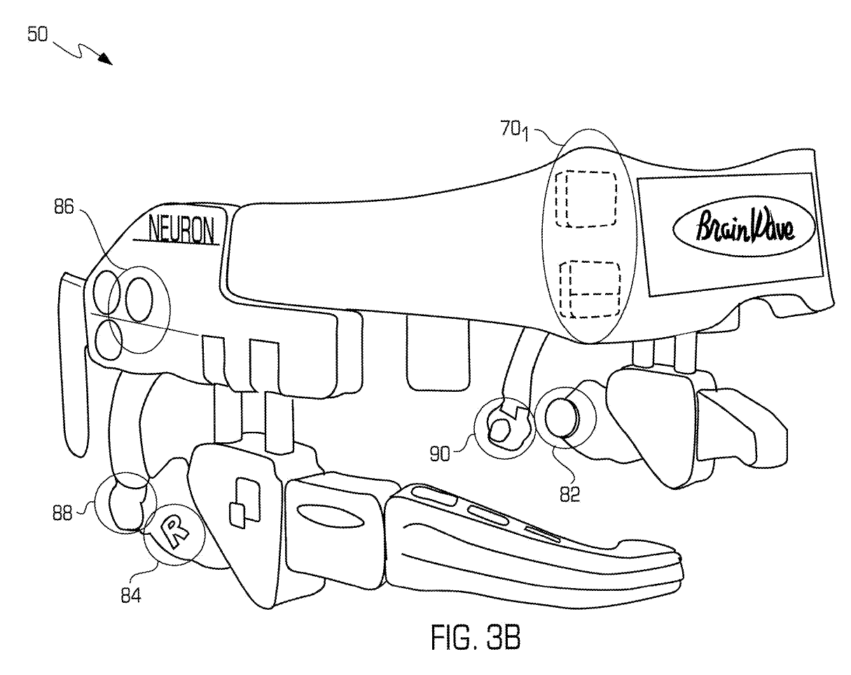

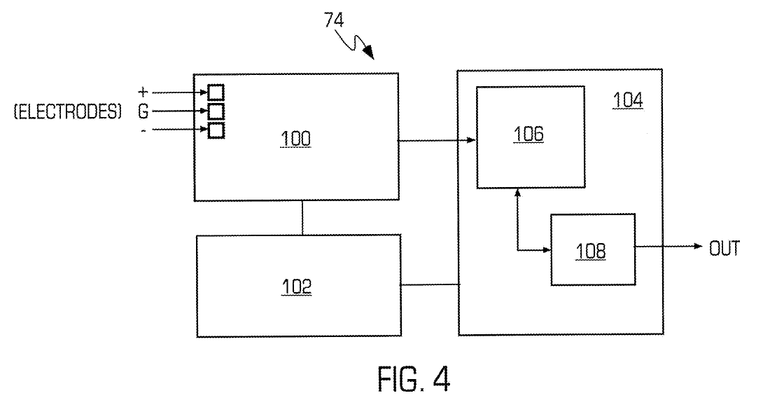

FIGS. 3A and 3B illustrate further details of the apparatus shown in FIGS. 1, 2A and 2B wherein FIG. 3A is a front view of the headset and FIG. 3B is a side perspective view of the headset. The headset may include one or more active dry sensors 70, such as a first set of active dry sensors 70₁ and a second set of active dry sensors 70₂, a Electrooculogram (EOG) up sensor 72 and a bio signal processing module 74 that are located on the front portion of the headset. The active dry sensors 70₁ and 70₂ measure the electroencephalogram (EEG) signals of the user of the headset. The EOG up sensor detects when the user of the headset is looking up. The EOG sensors detect EMG (electromyography) signals from muscles around eyes. To detect 4 directional movements of eyeball 4 EOG sensors are needed and each EOG sensor detects EMG signal of the small muscles when eyeball moves. In FIGS. 2 and 3, 3 EOG sensors are installed around the right eye and one sensor is installed left side of the left eye. The EOG sensor above the eye detect upward eyeball movement, while the sensor below the eye detects downward eyeball movement. The sensor at the right side of the eye detects EOG signal when the eyeball moves to right, and the sensor at the left side of the eye detects EOG signal when the eyeball moves to left. The bio signal processing module 74 processes the EEG and EOG signals detected by the sensors and generates a set of control signals. The bio signal processing module 74 is described in more detail with reference to FIG. 4.

There are generally two protocols to detect bio-signals; monopolar (unipolar) and bipolar. In the monopolar protocol, reference electrode is located where no bio signal is detected and there is no EEG signal at the backside of the ears or earlobe. Thus, for the monopolar protocol, the reference electrode is attached at the backside of the ear, while the active electrode is attached on the forehead. In the bipolar protocol, the reference electrode is attached where bio-signal (EEG signal) can be detected (generally one inch apart). For the bipolar protocol, both the active and reference electrodes are attached on the forehead. In the exemplary embodiment shown in FIGS. 3A and 3B, the monopolar protocol is used although the headset can also use the bipolar protocol in which both electrodes are attached on the forehead.

The headset may also include an EOG right sensor 76, an EOG down sensor 78 and an EOG left sensor 80 that detect when the user is looking right, down and left, respectively. Thus, using the four EOG sensors, the direction of eye movement while wearing the headset is determined which can be analyzed and used to generate the control signals that are used as a human/machine interface, etc. The headset 50 may further include a first speaker and a second speaker 82, 84 that fit into the ears of the user when the headset is worn to provide audio to the user. The headset may also include a power source 86, such as a battery, a ground connection 88 and a reference connection 90. The reference connection provides a baseline of the bio-signal the ground connection ensures a stable signal and protects the user of the headset. Thus, when the headset is worn by the user, the speakers fit into the ears of the user and the EEG and EOG signals from the user are detected (along with eye blinks) so that the headset in combination with other hardware and software is able to quantitatively evaluate the mental state of the user and then generate control signals (based in part of the mental state of the user) that can be used as part of a human/machine interface such as control signals used to control a toy as shown in FIG. 1.

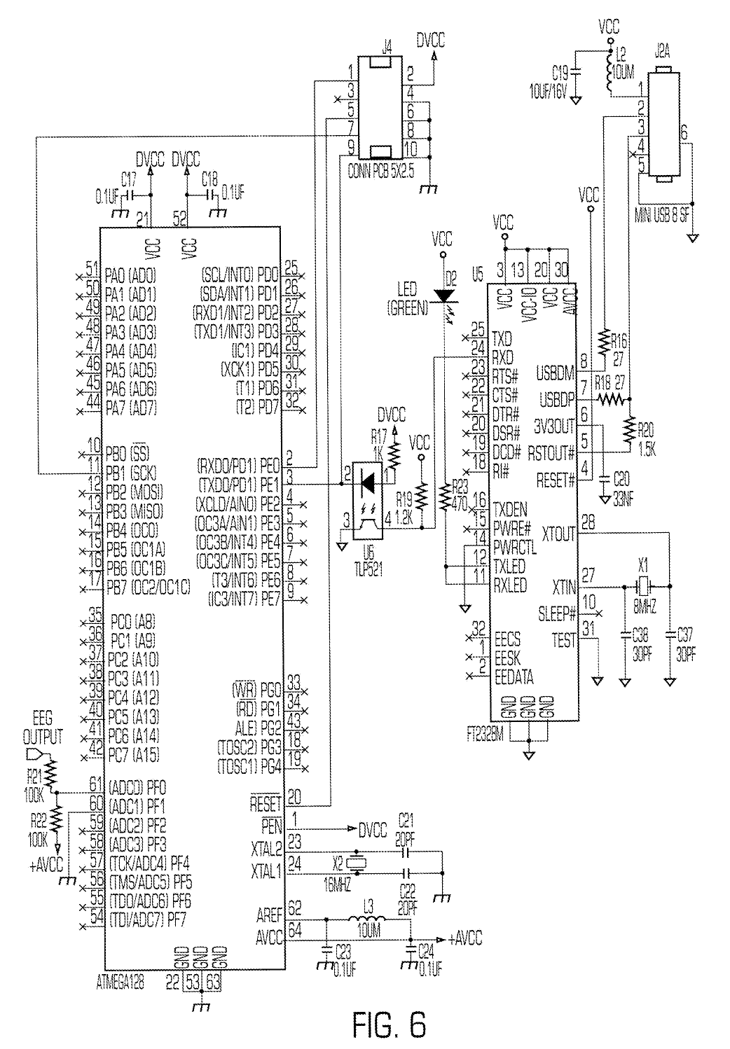

FIG. 4 illustrates an implementation of a system for controlling a toy using the apparatus for quantitatively evaluating mental states that includes the neuro headset shown in FIGS. 2A, 2B, 3A and 3B, other hardware and software. In particular, FIG. 4 shows an implementation of the bio processing module 74 in more detail wherein the module may include an analog part 100, a power supply/regulation part 102 and a digital part 104. The apparatus and method, however, are not limited to the particular hardware/software/firmware implementation shown in FIGS. 4-9. The analog part 100 of the module interfaces with the sensors and may include a positive, ground and negative inputs from the sensors. In some implementations, some portion of the analog portion may be integrated into the sensors that are part of the headset. The analog part may perform various analog operations, such as signal amplification, signal filtering (for example so that signals with a frequency range of 0 to 35 Hz are output to the digital part) and notch filtering and outputs the signals to the digital part 104. In an exemplary embodiment, the analog part may provide 10000× amplification, have an input impedance of 10T ohm, notch filtering at 60 Hz at −90 dB, provide a common mode rejection ratio (CMRR) of 135 dB at 60 Hz and provide band pass filtering from 0-35 Hz at −3 dB. The power supply/regulation part 102 performs various power regulation processes and generates power signals (from the power source such as a battery) for both the analog and digital parts of the module 74. In an exemplary embodiment, the power supply can receive power at approximately 12 volts and regulate the voltage. The digital part 104 may include a conversion and processing portion 106 that convert the signals from the analog part into digital signals and processes those digital signal to detect the mental state of the user and generate the output signals and a transmission portion 108 that transmits/communicates the generated output signals to a machine, such as the toys shown in FIG. 1, that can be controlled, influenced, etc. by the detected mental states of the user. The transmission portion may use various transmission protocols and transmission mediums, such as for example, a USB transmitter, an IR transmitter, an RF transmitter, a Bluetooth transmitter and other wired/wireless methods are used as interfaces between the system and machine (computer). In an exemplary embodiment, the conversion portion of the digital part may have a sampling rate of 128 KHz and a baud rate of 57600 bits per second and the processing portion of the digital part may perform noise filtering, fast fourier transform (FFT) analysis, perform the processing of the signals, generate the control signals and determine, using a series of steps, the mental state of the wearer of the headset. An exemplary circuit implementation of the processing portion and the transmission portion is shown in FIG. 6.

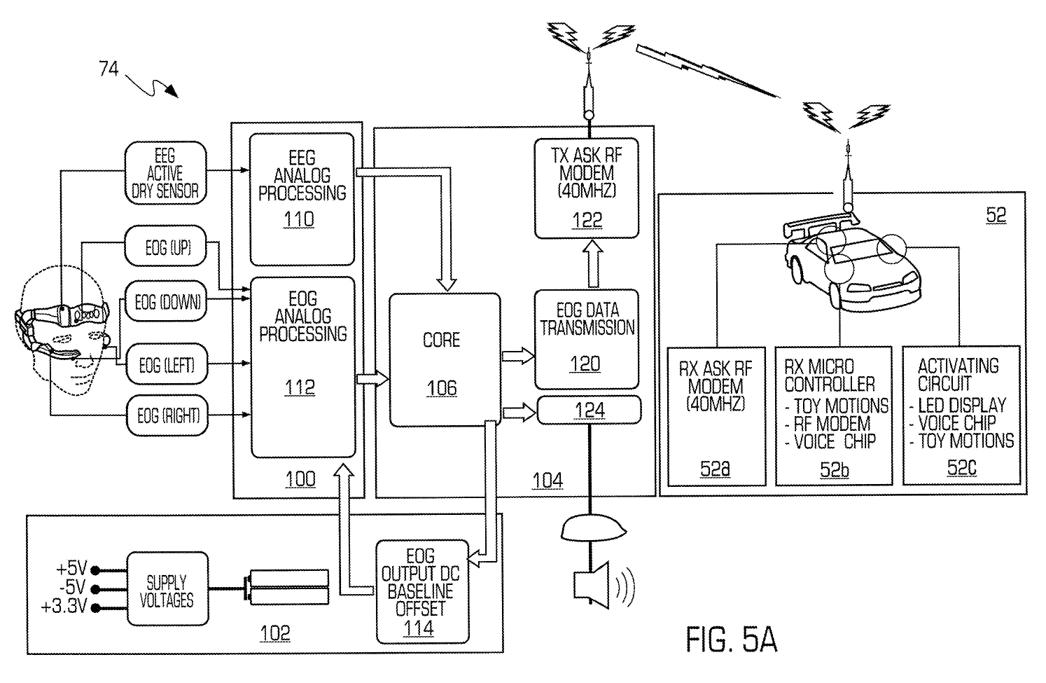

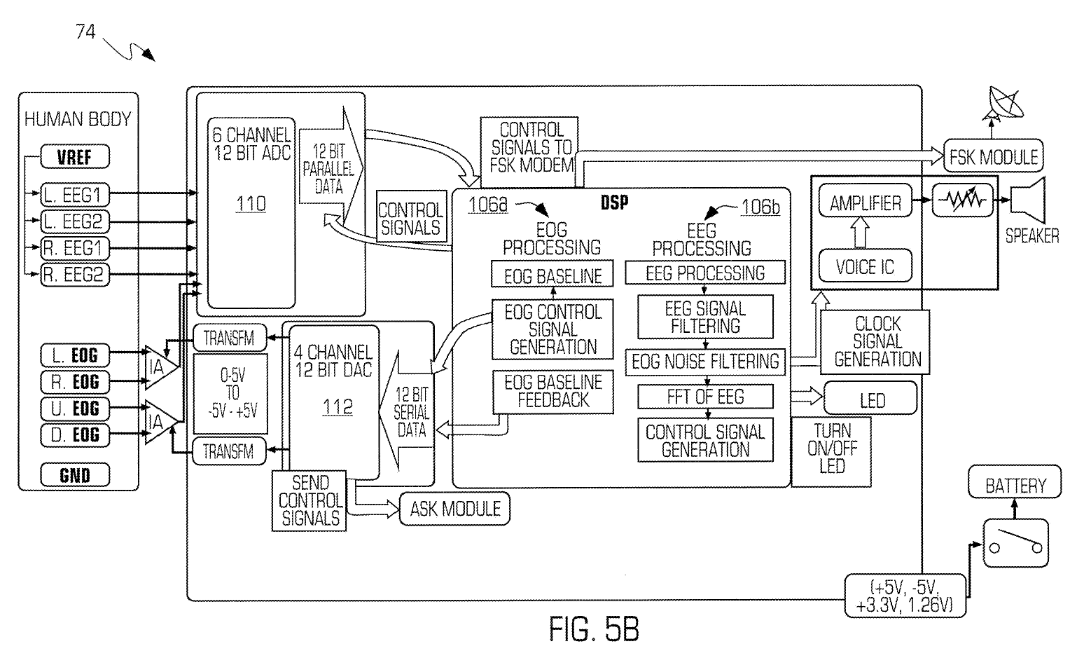

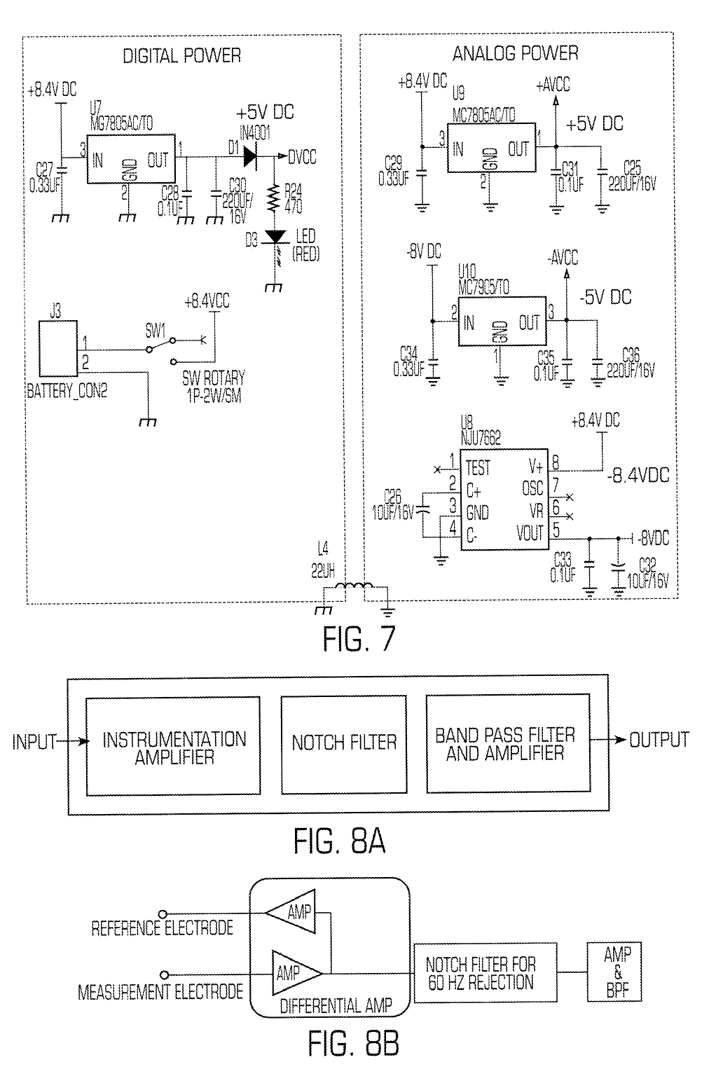

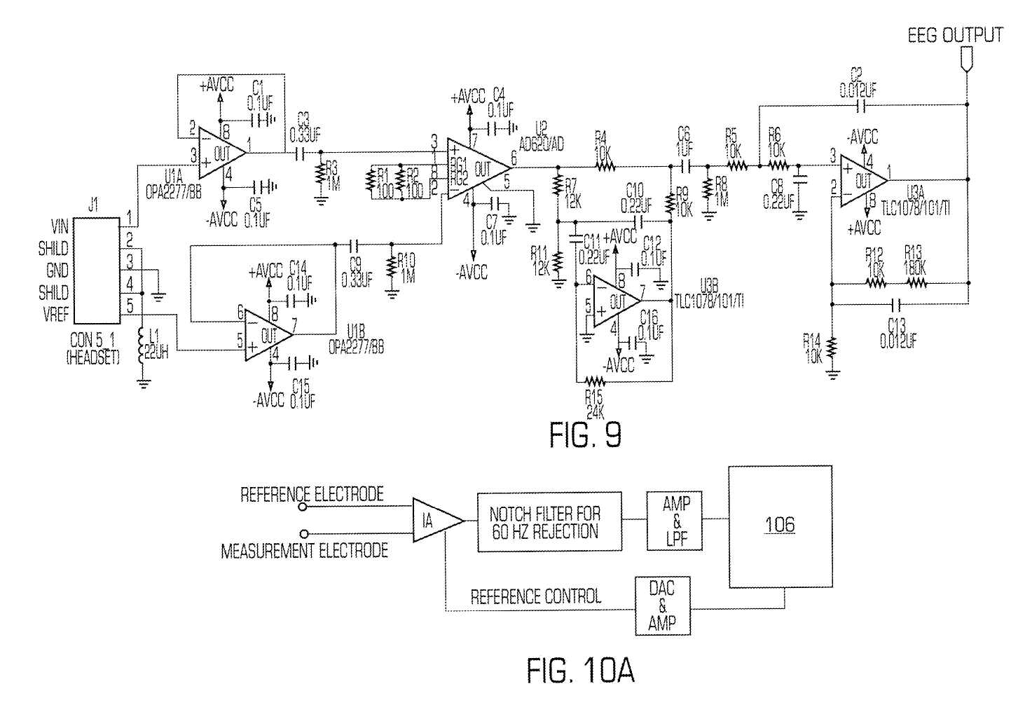

FIG. 5A illustrates more details of the hardware of the system shown in FIG. 4. In particular, the analog part 100 further comprises an EEG signal analog processing portion 110 (wherein the circuit implementation of this portion is shown in FIG. 9A) and an EOG analog processing portion 112 (wherein the circuit implementation of this portion is shown in FIG. 9B). The EOG processing portion may receive EOG output DC baseline offset signal from an EOG output DC baseline offset circuit 114. The EOG output DC baseline offset circuit 114 may be a shift register coupled to a processing core 106, a digital to analog converter coupled to the shift register and an amplifier that uses the analog signal output from the digital to analog converter to adjust the gain of an amplifier that adjusts the EOG signals. In an exemplary embodiment, the left and right EOG signals are offset using a first shift register, a first D/A converter and a first amplifier and the up and down EOG signals are offset using a second shift register, a second D/A converter and a second amplifier. The power regulation part 102 may generate several different voltages, such as +5V, −5V and +3.3V in the exemplary implementation wherein an exemplary circuit implementation of the power regulation part is shown in FIG. 7.

The digital portion 104 includes an analog to digital converter (not shown) and the processing core 106, that may be a digital signal processor in an exemplary embodiment with embedded code/microcode, that performs various signal processing operations on the EEG and EOG signals. In an exemplary embodiment, the analog to digital converter (ADC) may be a six channel ADC with a separate channel for each EEG signals, a channel for the combined left and right EOG signals (with the offset) and a channel for the combined up and down EOG signals (with the offset). In more detail, the signal may be sampled by an analog-to-digital converter (A/D converter) with sampling rate of 128 Hz and then the data are processed with specially designed routines so that the type of mental state of the user and its level are determined based on the data processing. These results are shown by numbers and graphically. The processing core may also generate one or more output signals that may be used for various purposes. For example, the output signals may be output to a data transmitter 120 and in turn to a communications device 122, such as a wireless RF modem in the exemplary embodiment, that communicates the output signal (that may be control signals) to the toy 52. The output signals may also control a sound and voice control device 124 that may, for example, generate a voice message to wake-up the user which is then sent through the speakers of the headset to provide an audible alarm to the user.

In the exemplary embodiment shown in FIG. 5, the communications device 122 is a 40 MHz RF amplitude shift key (ASK) modem that communicates with a 40 MHz RF ASK modem 52a in the toy. The toy also have a microcontroller 52b and an activating circuit 52c that allows the toy, based on the output signals communicated from the headset, to perform actions in response to the output signals, such as moving the toy in a direction, stopping the toy, changing the direction of travel of the toy, generating a sound, etc. In this exemplary embodiment, the apparatus with the headset replaces the typical remote control device and permits the user to control the toy with brain waves.

FIG. 5B illustrates more details of the hardware of the bio processing unit 74 of the system. The EEG and EOG analog processing units 110, 112 may be, in the exemplary embodiment, a six channel 12-bit analog to digital converter (ADC) to convert the analog EEG and EOG signals from the headset to digital signals and a four channel 12-bit digital to analog converter (DAC) to provide the feedback signals to the operational amplifiers for the EOG signals. The core 106 may further comprise an EOG processing unit 106a and a EEG processing unit 106b.

The EOG processing unit determines the EOG baseline signal and then generates the EOG control signals and also generates the EOG baseline feedback signals that are fed back to the operational amplifiers. The EOG baseline feedback and the EOG control signals are fed to the four channel 12-bit DAC as a 12 bit serial data channel. The EEG processing unit performs EEG signal filtering (described below in more detail), EOG noise filtering of the EEG signals (described below) and perform the fast fourier transform (FFT) of the EEG signals. From the FFT transformed EEG signals, the EEG processing unit generates the control signals.

FIG. 6 illustrates an exemplary circuit implementation of the digital portion of the hardware shown in FIG. 4. The processing core, in this exemplary implementation, is a ATmega128 that is a low-power CMOS 8-bit microcontroller based on the AVR enhanced RISC architecture which is commercially sold by Atmel Corporation with further details of the particular chip available at http://www.atmel.com/dyn/resources/prod_documents/doc2467.pdf which is incorporated herein by reference. The transmission circuit is FT232BM which is a USB UART chip that is commercially available from Future Technology Devices International Ltd. and further details of this chip are http://www.ftdichip.com/Products/FT232BM.htm which is incorporated herein by reference.

FIG. 7 illustrates an exemplary circuit implementation of the power regulation portion of the hardware shown in FIG. 4. In particular, the analog and digital power portions of the apparatus are shown.

FIG. 8A illustrates more details of an analog portion of each dry-active electrodes wherein each electrode/sensor includes instrumentation amplification, a notch filter and a band pass filter and amplifier. As shown in FIG. 8B, each dry-active electrode/sensor has a reference electrode and a measurement electrode that are connected to a differential amplifier (formed using two operational amplifiers connected together in a known manner) whose output is coupled to the notch filter that rejects 60 Hz signals (power line signals) and then the output of the notch filter is coupled to the bandpass filter and amplifier.

FIG. 9 illustrates an exemplary circuit implementation of the analog EEG signal processing portion of the hardware shown in FIG. 5 that performs the analog processing of the EEG signals generated by the EEG sensors of the apparatus. As shown, the circuit uses one or more amplifiers in order to process and amplify the EEG signals of the apparatus.

FIG. 10A is a block diagram of the analog EOG signal processing portion shown in FIG. 5 and FIG. 10B illustrates an exemplary circuit implementation of the analog EOG signal processing portion shown in FIG. 5. As shown in FIG. 10A, the analog EOG signal processing portion receives a reference electrode signal and a measurement electrode signal that are fed into an amplifier whose gain/offset is adjusted by the reference control signal generated by the processing core 106 through the DAC and the amplifier. The output of the amplifier is fed into a notch filter (to reject 60 Hz signals from power lines) which is then fed into an amplifier and low pass filter before being fed into the processing core 106. FIG. 10B illustrates the exemplary circuit implementation of the analog EOG signal processing portion wherein one or more operational amplifiers perform the signal processing of the EOG signals.

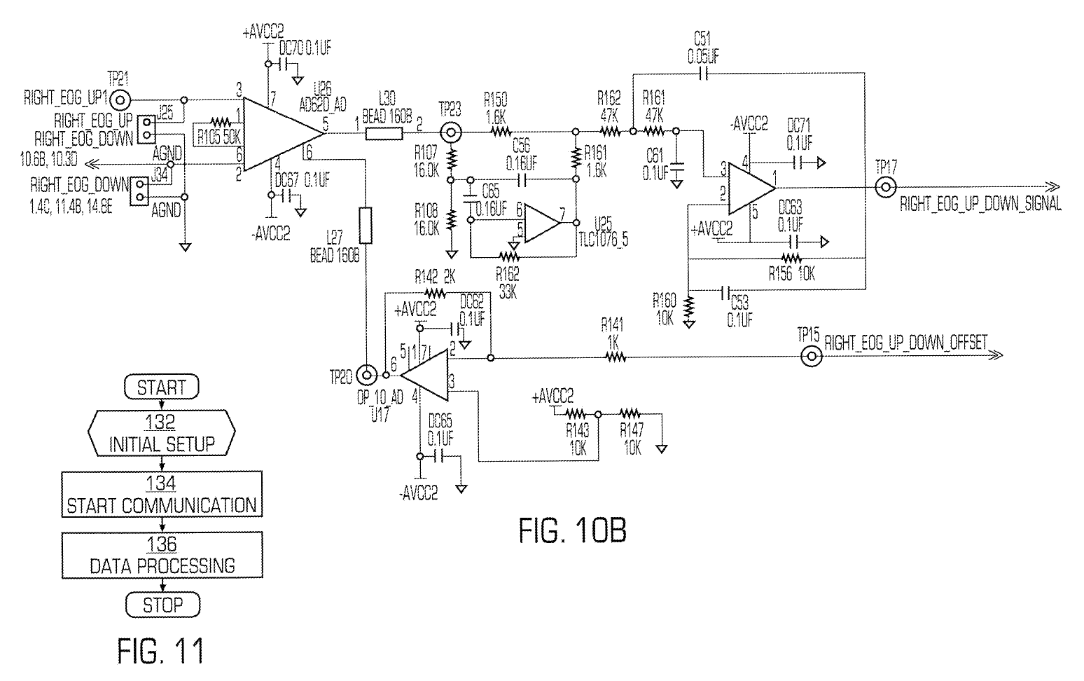

FIG. 11 illustrates an example of the operation of the software 130 that is part of the shown in FIG. 4. An initial setup (132) begins the operation of the software of the apparatus. Once the initial setup is completed, a communication session with the object being controlled is started (134). Once the communications are started, the software performs the signal processing of the electrode signals and the data processing of the digital representation of the EEG and EOG signals.

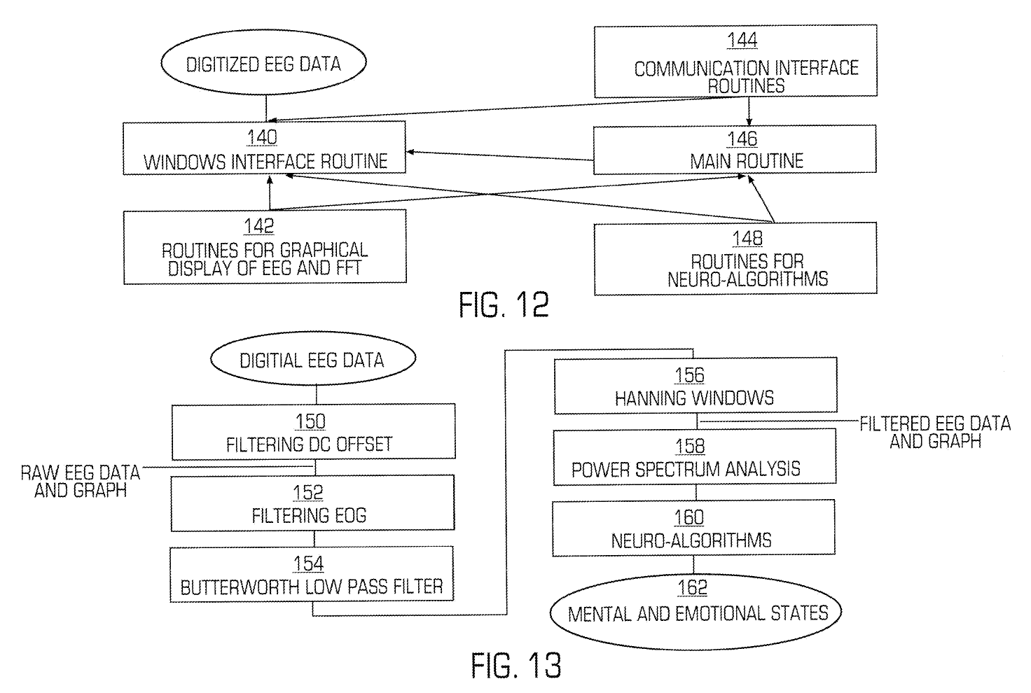

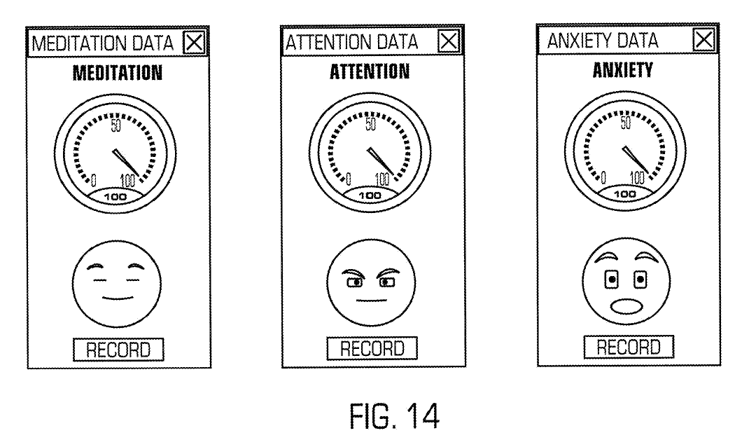

FIG. 12 illustrates further details of the data processing process of FIG. 11 wherein the data processing process includes a plurality of routines wherein each routine is a plurality of lines of computer code (implemented in the C or C++ language in the exemplary embodiment) that may be executed by a processing unit such as embedded code executed by the processing core 106 shown in FIG. 5 or on a separate computer system. The process may include a Windows interface routine 140, a routine 142 for the graphical display of the EEG and FFT signals, a routine 144 for the communications interface, a main routine 146 and a neuro-algorithm routine 148. The main routine controls the other routines, the Windows interface routine permits the data processing software to interface with an operating system, such as Windows and the routines 142 generate a graphical display of the EEG and FFT signals. The communications routine 144 manages the communications between the apparatus and the object being controlled using the apparatus and the neuro-algorithm routine processes the EEG and EOG signals to generate the control signals and generate a graphical representation of the mental state of the user of the apparatus as shown in FIG. 14.

The mental state of the user, once measured, can be placed into a level scale such as a level from 0 to 100 as shown in FIG. 14. The mental state (and the measured level of the mental state) of the user may be used to generate control signals to control a machine, such as a computer. The control of the machine may include cursor or object movement at video displays (wherein a high level of a mental state the cursor or object moved upward or faster or vice versa), volume control of speakers (wherein a high level of the mental state increases the volume and vice versa), motion control of the machine (wherein a high level of the mental state causes the machine to move faster and vice versa), selecting music (songs) in portable audio system, including mp3 (wherein a piece of music or a song of a specific genre and tempo of the stored music or songs are selected is the song/music matches the mental state and the level of the mental state), biofeedback or neurofeedback that can be used for mental training, such as relaxation or attention training or may be useful to test stress level, mental concentration level and drowsiness), and/or other brain-machine (computer) interfaces such as on/off control, speed control, direction control, brightness control, loudness control, color control, etc.

FIG. 13 illustrates a flowchart 150 of the data processing steps. First, the DC offset of the digital EEG data is filtered out (150) so that the raw EEG data can be graphically displayed and the EOG signals can be filtered (152). The EOG signals may be filtered using the known JADE algorithm to filter noise. Then, the EEG and EOG signals are low pass filtered (154) and then the signals are Hanning windowed (156). The filtered EEG data signals are generated and can be graphed. Then, the filtered signals are analyzed for their power spectrum (158) which are then fed into the neuro-algorithms (160) so that the mental and emotional states of the user (162) are determined. The power spectrum analysis is performed for 512 data point at every second. Using the power spectrum analysis, the power spectrum data for the delta, theta, alpha and beta waves are extracted.

The neuro-algorithm, which consists of several equations and routines, computes levels of mental states using the power spectrum data of the delta, theta, alpha and beta waves. These equations are made based on a data base of experiments. These equations can be modified and changed for different applications and user levels. The mental state can be expressed as attention, relaxation or meditation, anxiety and drowsiness. Each mental state level is determined by the equation which includes delta, theta, alpha and beta power spectrum values as input data. The level of the mental state can be represented by the number from 0 to 100, which may be changed depending on applications. The value of mental state level is renewed every second. Then, the mental and emotional states may be used by the apparatus to, for example, generate the control signals or display the mental states of the user as shown in FIG. 14.

The apparatus, as described above, measures the EEG (two channels) and EOG signals (four channels) of the user as well as eye blinks. Using the apparatus, the mental state of the user can be determined as shown in the following table:

MENTAL STATES OF USER

| | Occupied frequency | |
|---|---|---|
| EEG type | bandwidth | Mental states & conditions |
| Delta | 0.1 Hz~3 Hz | deep, dreamless sleep, non-REM |
| | | sleep, unconscious |
| Theta | 4 Hz~7 Hz | intuitive, creative, recall, fantasy, |
| | | imagery, creative, dreamlike, |
| | | switching thoughts, drowsy |
| Alpha | 8 Hz~12 Hz | eyes closed, relaxed, not agitated, |
| | | but not drowsy, tranquil conscious |
| Low Beta | 12 Hz~15 Hz | formerly SMR, relaxed yet |
| Midrange Beta | 16 Hz~20 Hz | focused, integrated thinking, |
| | | aware of self & surrounding |
| High Beta | 21 Hz~30 Hz | alertness, agitation |

In an exemplary implementation of the system, the EEG sensors may be gold plate, dry sensor active electronic circuits wherein each EEG sensor may include amplification and band pass filtering. The EEG sensor module may have a gain of 80 dB and a bandpass filter bandwidth of 1 Hz-33 Hz at −1 dB, 0.5 Hz-40 Hz at −3 dB and 0.16 Hz-60 Hz at −12 dB. Each EOG sensor may be a gold plate passive sensor and may have a gain of 60 dB with a low pass filtering bandwidth of DC −40 Hz at −1 dB. The wireless communication mechanism may be a 27 or 40 MHz ASK system, but may also be a 2.4 GHz ISM communications method (FHSS or DSSS). The analog to digital conversion may be 12 bits and the sampling frequency may be 128 Hz. The total current consumption for the apparatus is 70 mA at 5 VDC and the main power supply is preferably DC 10.8V, 2000 mAh Li-Ion rechargeable battery.

While the foregoing has been with reference to a particular embodiment of the invention, it will be appreciated by those skilled in the art that changes in this embodiment may be made without departing from the principles and spirit of the invention, the scope of which is defined by the appended claims.

1. An apparatus for determining the mental state of a user, the apparatus comprising:
a frame;
one or more dry-active sensors located on the frame that are capable of detecting the brain waves of a user when the sensors touch a skin portion of a user and of generating brain wave signals; and
a processing unit that receives the brain wave signals, processes the brain wave signals and generates a signal corresponding to a level of a mental state of the user.

2. The apparatus of claim 1, wherein the processing unit further comprises an analog processing portion that converts the brain wave signals into a set of digital brain wave signals and a digital processing portion that processes the digital brain wave signals to generate the signal corresponding to the level of the mental state of the user.

3. The apparatus of claim 2, wherein the analog processing portion further comprises an analog-to-digital converter and wherein the digital processing portion further comprises a processing core, a memory that stores one or more routines for processing the digital brain wave signals wherein the routines are executed by the processing core and an output interface that outputs the signal corresponding to the level of the mental state of the user.

4. The apparatus of claim 3, wherein the processing core generates a control signal based on the signal corresponding to the level of the mental state of the user and wherein the output interface further comprises a data transmission unit that transmits the control signal to a remote object that is controlled based on the control signal.

5. The apparatus of claim 4, wherein the remote object further comprises one of a video display, a speaker, a machine, a portable audio device and a computer.

6. The apparatus of claim 5, wherein the control signal controls a cursor of the video display.

7. The apparatus of claim 5, wherein the control signal controls a volume of the speaker.

8. The apparatus of claim 5, wherein the control signal controls a speed of motion of the machine.

9. The apparatus of claim 5. wherein the control signal controls a piece of music selected on the portable audio device.

10. The apparatus of claim 5, wherein the control signal controls one of neurofeedback and biofeedback provided to the user by the computer.

11. The apparatus of claim 5, wherein the control signal controls one of an on/off selection, a speed control, a direction control, a brightness control, a loudness control and a color control of the computer.

12. The apparatus of claim 3, wherein the one or more routines further comprises a routine for evaluating a mental state of the user based on the digital brain wave signals wherein the routine is a plurality of lines of computer code executed by the processing core.

13. The apparatus of claim 1 further comprises a processing core and a memory that stores one or more routines for processing the digital brain wave signals wherein the routines are executed by the processing core.

14. The apparatus of the claim 2 further comprises a power supply unit that supplies power to the analog processing portion and the digital processing portion.

15. The apparatus of claim 1, wherein the frame has a front portion, a first side portion attached to the front portion and a second side portion opposite of the first side portion, and wherein the one or more dry-active sensors are located on the front portion of the frame that contacts a forehead of the user and are located on the first and second side portions of the frame.

16. The apparatus of claim 15, wherein each dry-active sensor further comprises a mechanical portion that interfaces with a user and an electronic portion having an amplifier circuit and a filter circuit that outputs a filters brain wave signal.

17. The apparatus of claim 4, wherein the data transmission unit further comprises a universal serial bus transmission unit, an infrared transmission unit, a radio frequency transmission unit, a Bluetooth transmission unit, a wireless transmission unit or a wired transmission unit.

18. The apparatus of claim 15, wherein the one or more dry-active sensors are in a monopolar protocol.

19. The apparatus of claim 1, wherein the frame has a front portion, a first side portion attached to the front portion and a second side portion opposite of the first side portion, and wherein the one or more dry-active sensors are located on the front portion of the frame that contacts a forehead of the user and the one or more dry-active sensors are in a bipolar protocol.

20. A method for determining the mental state of a user, the method comprising:
detecting, using one or more dry-active sensors located on the frame, a set of brain wave signals of a user when the sensors touch a skin portion of a user; and
receiving, at a processing unit, the set of brain wave signals; and
processing, in the processing unit, the brain wave signals to generates a signal corresponding to a level of a mental state of the user.

21. The method of claim 20, wherein processing the brain wave signals further comprises converting, using an analog processing portion, the brain wave signals into a set of digital brain wave signals and processing, using a digital processing portion, the digital brain wave signals to generate the signal corresponding to the level of the mental state of the user.

22. The method of claim 20 further comprising generating, in the processing unit, a control signal based on the signal corresponding to the level of the mental state of the user, transmitting, using a data transmission unit, the control signal to a remote object and controlling the remote object based on the control signal.

23. The method of claim 22, wherein controlling the remote object based on the control signal further comprises controlling a cursor of the video display based on the control signal.

24. The method of claim 22, wherein controlling the remote object based on the control signal further comprises controlling a volume of a speaker based on the control signal.

25. The method of claim 22, wherein controlling the remote object based on the control signal further comprises controlling a speed of motion of the machine based on the control signal.

26. The method of claim 22, wherein controlling the remote object based on the control signal further comprises selecting a piece of music on a portable audio device based on the control signal.

27. The method of claim 22, wherein controlling the remote object based on the control signal further comprises generating one of neurofeedback and biofeedback based on the control signal.

28. The method of claim 22, wherein controlling the remote object based on the control signal further comprises one of selecting an on/off selection, selecting a speed level, selecting a direction, selecting a brightness level, selecting a loudness level and selecting a color level.

29. The method of claim 22, wherein transmitting the control signal to a remote object further comprises one of transmitting the control signal using a universal serial bus transmission unit, transmitting the control signal using an infrared transmission unit, transmitting the control signal using a radio frequency transmission unit, transmitting the control signal using a Bluetooth transmission unit, transmitting the control signal using a wireless transmission unit and transmitting the control signal using a wired transmission unit.

30. The method of claim 20, wherein the detecting a set of brain waves signals further comprises, detecting, using one or more dry-active sensors in a monopolar protocol, the set of brain waves signals of a user when the sensors touch a skin portion of a user.

31. The method of claim 20, wherein the detecting a set of brain waves signals further comprises, detecting, using one or more dry-active sensors in a bipolar protocol, the set of brain waves signals of a user when the sensors touch a skin portion of a user.
